(19) United States
(12) Patent Application Publication
(10) Pub. No.: US 2012/0220976 A1
(43) Pub. Date: Aug. 30, 2012

(54) Absorbent Washable Boxer Shorts

(76) Inventors: Kize Morse, Canyon, TX (US); Mae Morse, Canyon, TX (US)

(21) Appl. No.: 13/035,065

(22) Filed: Feb. 25, 2011

(51) Int. Cl. A61F 13/496

(52) U.S. Cl. 604/385.15; 604/385.01

(57) ABSTRACT

An absorbent undergarment for men comprising: a waistband; two openings for the legs of the user; a slit in a front portion of the undergarment; and an absorbent pad on the inner surface of the rear portion of the undergarment. The pad is permanently stinted on the inner surface of the undergarment. The pad and undergarment according to the present invention may be made of washable and reusable materials.

### BACKGROUND OF THE INVENTION

1. Field of Invention

The field of invention relates to absorbent washable boxer shorts that are utilized for men.

2. Description of Related Art

Undergarments is clothing that's worn directly against the skin underneath the principal clothing of the individual. Women wear undergarments such as panties, bras and or slips under their clothing to protect their clothing from body fluids that may emit during wear. Men also utilize undergarments such as briefs, boxer shorts and undershirts. Soiling of the undergarments is a consequence of their use. Many undergarments are made of cotton, nylon or polyester materials and absorb any minor body fluids that may be emitted by the user. Additional padding or absorbent material has been developed to utilize with undergarments or to be stitched into undergarments to absorb more significant body fluid leakage that may occur during use.

Most of the additional padding or absorbent material used in undergarments relates to padding made for women undergarment that collect the fluid discharges that may occur at unexpected times, i.e., preceding and during menstrual periods. Women may utilize panty pads or napkins that may attach to the undergarment to absorb the leakage of bodily fluids. Some men also use incontinence padding to absorb any urine that may leak unexpectedly. An example of an incontinence or panty liner is described in U.S. Pat. No. 6,013,066, which discloses an absorbent article such as a sanitary napkin or pad for persons afflicted with light incontinence to be worn in the crotch part of the underwear. Disposable incontinence undergarments address more significant body fluids such as described in U.S. Pat. No. 6,984,279, which discloses a method of making an undergarment having a front and back crotch seam which includes a flat web. The flat web provides a front-to-back crotch seam of absorbent material. U.S. Pat. No. 7,086,095 discloses a boxer-style absorbent pants that includes side-to-side and side seams from a flat web where the flat web is folded and provides crotch protection for incontinence.

The drawbacks the padded undergarments of the prior art include the focus on the protection for crotch or frontal leakage. Further much of the padding is disposable in that the padding is inserted into the existing undergarments or undergarments made specifically for disposable purposes. Therefore it would be advantageous to have an undergarment that is utilized or includes padding in the buttocks area for leakage, which is washable and reusable. This padding would be especially designed for boxer-style shorts worn by men.

### SUMMARY OF THE INVENTION

The present invention relates to an absorbent undergarment for men comprising: a waist band; two openings for the legs of the user; a slit in a front portion of the undergarment; and an absorbent pad on the inner surface of the rear portion of the undergarment. The pad is permanently stinted on the inner surface of the undergarment. The pad and undergarment according to the present invention may be made of washable and reusable materials.

### BRIEF DESCRIPTION OF DRAWINGS

In reference to FIG. 1 a pair of boxer shorts including rear padding in accordance with the present invention are depicted.

FIG. 2 depicts the rear view of the boxer shorts according to the present invention with the inclusion of absorbent padding.

### DETAILED DESCRIPTION

The present invention relates to a pair of washable boxer shorts that includes sufficient padding for any leakage that may occur and stain the buttocks area of the boxer shorts. The absorption of moisture in this area helps to prevent stains onto the user's outergarments while wearing the boxer shorts in accordance with the present invention.

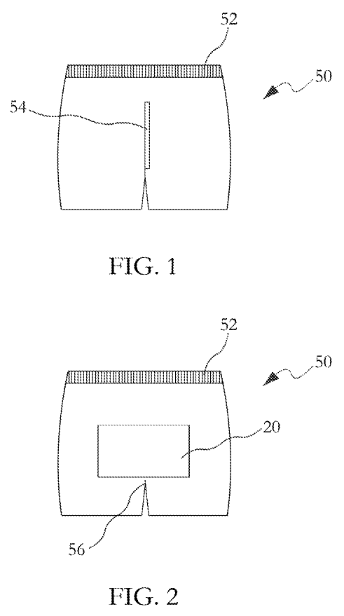

In reference to FIG. 1, a front view of a pair of Boxer Shorts 50 according to the present invention is depicted. The Boxer Shorts 50 includes a Waistband 52 and a Slit 54 on the front portion of the Boxer Shorts 50. Boxer shorts 50 have openings for each leg of the individual wearing the boxer shorts. The boxer shorts 50 according to the present invention include paddings on the inside rear portion of the garment as depicted in FIG. 2. The Boxer Shorts 50 include an Absorbent Pad 20 located in the center portion of the rear of the Boxer Shorts 50. The Boxer Shorts 50 include a Center Seam 56 as shown in FIG. 2. The Absorbent Pad 20 is placed just above the Center Seam 56 along the inner surface of the rear portion of the Boxer Shorts 50. This Absorbent Pad 20 remains in place during use and is permanently stitched into the Boxer Shorts 50. The Boxer Shorts 50 are therefore reusable and washable so that the user may wash these shorts for reuse at a later date.

The Boxer Shorts 50 according to the present invention include the embedded absorbent padding as an alternative option for protection against accidental leakage. This additional padding absorbs moisture or bodily fluids that may be seeping and therefore provides better protection for the outergarments of the wearer of the boxer shorts. Further these boxer shorts may be reused due to the permanent stitching of the padding and the fabric used is suitable for multiple washing and reuse by the user.

What is claimed is:

1. An absorbent undergarment for men comprising:
a. a waistband;
b. two openings for the legs of the user;
c. a slit in a front portion of the undergarment; and
d. an absorbent pad on the inner surface of the rear portion of the undergarment.

2. The absorbent undergarment according to claim 1, where the pad is permanently stinted on the inner surface of the undergarment.

3. The absorbent undergarment according to claim 1, where the pad and undergarment are made of washable and reusable materials.
